(19) United States
(12) Patent Application Publication
(10) Pub. No.: US 2026/0199692 A1
(43) Pub. Date: Jul. 16, 2026

(54) FEEDTHROUGH PIN ASSEMBLY FOR ELECTRICAL CONNECTION IN AN IMPLANTABLE MEDICAL DEVICE

(71) Applicant: Medtronic, Inc., Minneapolis, MN (US)

(72) Inventors: Hailiang Zhao, Plymouth, MN (US); Sadegh Behdad, Apple Valley, MN (US)

(21) Appl. No.: 19/134,251

(22) PCT Filed: Nov. 9, 2023
(86) PCT No.: PCT/IB2023/061322

§ 371 Date: May 30, 2025

Related U.S. Application Data
(60) Provisional application No. 63/386,622, filed on Dec. 8, 2022.

(51) Int. Cl. A61N 1/375; A61L 31/02; A61L 31/04; A61N 1/378

(52) U.S. Cl. CPC A61N 1/3754; A61L 31/022; A61L 31/04; A61N 1/3758; A61N 1/378

(57) ABSTRACT

A feedthrough pin assembly for a medical device including a feedthrough pin having a distal portion configured for insertion through an opening in a battery cover of a battery housing. The feedthrough pin assembly may also include a current collector having a tab configured to electrically couple, using resistance welding, to the distal portion of the feedthrough pin extending from a distal surface of the battery cover. The feedthrough pin assembly may also include a proximal portion of the feedthrough pin configured to be secured to a proximal surface of the battery cover including orienting a portion of the proximal end to contact the proximal surface of the battery cover. The battery cover may be configured to attach to a battery container, enclosing a battery configured to contact the current collector, wherein the battery container is coupled to the feedthrough pin and current collector.

This application claims the benefit of U.S. Provisional Patent Application Ser. No. 63/386,622, filed 8 Dec. 2022, the entire content of which is incorporated herein by reference.

### TECHNICAL FIELD

This disclosure relates to implantable medical devices and to feedthrough connections between components of a medical device.

### BACKGROUND

Some types of implantable medical devices (IMDs), cardiac pacemakers or implantable cardioverter defibrillators systems, or other IMDs, may be used to provide cardiac sensing and therapy for a patient via one or more electrodes. Some IMDs include one or more feedthrough pins used for a variety of purposes in an implantable medical device. Some IMDs use feedthrough pins as electrical connections to terminals of a hermetically sealed battery in the IMD.

The feedthrough pins may be used to electrically connect an internal part of an IMD with an external component. In some examples, a feedthrough pin may be used to electrically connect the internal electrodes of a hermetically sealed battery to an external circuit or harness. When using feedthrough pins to electrically connect components, an IMD may benefit from the surrounding feedthrough pins with an insulator to prevent the feedthrough pin from inadvertently grounding on other metal components. Using a metallic battery case may allow the machined metal housings for IMD batteries, to be less complex. However, a metallic battery case is electrically conductive and may cause electrical shorting of the feedthrough pin to the battery case or housing. While use of a polymeric battery housing may limit the risk of electrical shorting, new challenges arise when utilizing hermitic sealing techniques on a polymeric battery housing.

### SUMMARY

In accordance with some of the techniques of the disclosure, a feedthrough pin assembly for use in a medical device is set forth herein. A feedthrough pin, partially disposed within a battery housing, may electrically connect an electrode of the battery to electrical components outside the battery housing. Hermetic seals applied between the feedthrough pin and the battery cover, and between the battery cover and the rest of the battery housing, may hermetically seal a battery with the battery housing.

The disclosure describes systems, techniques, and devices for medical device implantation systems (e.g., systems for device implantation via a vascular system of a patient). The present disclosure describes examples of feedthrough pin assemblies, and manufacturing techniques for use in IMDs. The feedthrough pin assembly may include a polymer battery cover, which may be a part of the battery housing. The battery cover may connect to a battery canister once coupled to a feedthrough pin. In some examples, the battery housing may be hermetically sealed by hermetically sealing the battery cover to the rest of the battery housing and by sealing the feedthrough pin to the battery cover by sealing a junction or gap between the feedthrough pin and the batter cover.

In one example, a technique of utilizing a feedthrough pin assembly including inserting a distal portion of a feedthrough pin through an opening in an battery cover; electrically coupling, using resistance welding, a tab of a current collector to the distal portion of the feedthrough pin extending from a distal surface of the battery cover; securing a proximal portion of the feedthrough pin to a proximal surface of the battery cover including orienting a portion of the proximal end to contact the proximal surface of the battery cover; and attaching the battery cover, the battery cover being mechanically coupled to the feedthrough pin, to a battery container, wherein the battery container is configured to hold a battery terminal in contact with the current collector coupled to the feedthrough pin.

In another example, a feedthrough pin assembly for a medical device including a feedthrough pin including a distal portion configured for insertion through an opening in an battery cover of a battery housing; a current collector including a tab configured to electrically couple, using resistance welding, to the distal portion of the feedthrough pin extending from a distal surface of the battery cover; a proximal portion of the feedthrough pin configured to be secured to a proximal surface of the battery cover including orienting a portion of the proximal end to contact the proximal surface of the battery cover; the battery cover configured to attach to a battery container, enclosing a battery configured to contact the current collector, wherein the battery container is coupled to the feedthrough pin and current collector; wherein the battery cover, being mechanically coupled to the feedthrough pin, wherein the battery container is configured to attach to the battery cover, wherein the battery container is configured to hold a battery terminal in contact with the current collector coupled to the feedthrough pin.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual drawing illustrating an example of a hermetically sealed Implantable Medical Device (IMD) implanted subcutaneously within a patient.

FIG. 2 is a conceptual drawing illustrating a feedthrough pin assembly connected to an IMD battery, in accordance with one or more techniques of this disclosure.

FIG. 3 is a conceptual drawing illustrating a disassembled closeup of a feedthrough pin assembly coupled to battery cover, in accordance with one or more techniques of this disclosure.

FIG. 4 is a conceptual drawing illustrating the manufacturing process of creating a feedthrough pin assembly within a hermetically sealed housing, in accordance with one or more techniques of this disclosure.

FIG. 5 is a flow chart illustrating a technique 500 of utilizing a feedthrough pin assembly accordance with one or more techniques of the disclosure.

### DETAILED DESCRIPTION

The disclosure describes implantable medical devices (IMDs), including electrical connections pins, and the assembly thereof, configured to interconnect electrical components within IMDs and associated techniques, structures, and assemblies configured to provide electrical connections within medical devices that have been hermetically sealed and implanted within a patient. An IMD may include a feedthrough pin assembly configured to electrically connect components enclosed in a hermetically sealed housing with components external to the housing. Some components may include battery components and the hermetically sealed housing may include a battery housing. The feedthrough pin assembly may be coupled to the components of the battery enclosed by the hermetically sealed battery housing. The feedthrough assembly may be configured to route electrical currents produced by the battery to other electrical components of the IMD located externally to the hermetically sealed battery housing. The battery may be one of a variety of energy sources including, a lithium-ion battery, a silver-oxide battery, a lithium polymer battery, a super capacitor, a hydrogen cell, or other stored electrical energy source that may be incorporated into an IMD.

In some examples, the feedthrough assembly may include an elongated pin including a distal end partially disposed within a proximal end of a hermetically sealed battery housing. In some cases, the proximal end of the hermetically sealed battery housing may include a battery cover. The battery cover may be partially or completely constructed of a plastic. Some examples of a plastic may include, polyimide, nylon, acrylic, and any other medical grade polymer. The distal end of the feedthrough pin may be bent at about a ninety-degree angle and electrically connected to a current collector of the battery. In some examples, a proximal end of the feedthrough pin may be exposed from the battery housing. The proximal end may be electrically connected to an electrical harness, electrical components, electrical circuit board, or other electrical components external to the battery housing.

The battery cover or proximal end of the battery housing, used for a hermetically sealed battery inside an IMD, may impact the feedthrough pin assembly used to maintain a hermetically sealed housing for the IMD battery. A metallic battery housing may include a large opening configured to accommodate a feedthrough pin and an electrical insulator. The insulator may be circumferentially formed around the feedthrough pin to prevent electrical shorting between the feedthrough pin and a rim of the opening of the metallic battery housing. The metallic housing may take more time and precision to machine while making sure the rim of the opening for the feedthrough pin is not sharp enough to wear through the insulator. In contrast, a plastic battery cover, used in a hermetically sealed battery housing may be configured with a small opening for which the feedthrough pin may be disposed into the battery housing. The electrical insulative nature of the plastic battery cover may allow the use of a conductive feedthrough pin without also using a circumferentially formed insulator. The plastic battery cover also allows for the use of a variety of sealing methods, to hermetically seal a gap formed between the feedthrough pin and the rim of the opening in the battery cover. Because the battery cover is plastic, sealing methods may include epoxy, curing resin, or plastic welding may be used between the metal feedthrough pin and the battery cover.

Assembly of the hermetically sealed housing around the IMD battery may be facilitated with the use of a plastic battery cover. In some examples, the plastic battery cover may be completely made of plastic or include a portion made of plastic. In one example, an opening in the plastic battery cover for receiving the feedthrough pin may be formed in the plastic portion of the battery cover. In some examples, the opening may be formed by applying compression force to the proximate end of a feedthrough pin resulting in a distal end of the feedthrough pin puncturing the plastic portion of the battery cover for the battery housing.

In some examples, the distal end of the feedthrough pin may be electrically connected to the current collector with a resistance weld. Resistance welding may include a process where a first welding electrode is connected to the current collector and a second welding electrode is connected to the proximate end of the feedthrough pin. A welding device supplies a high level of electrical current between the two welding electrodes. The current will pass through the low resistance feedthrough pin and current collector without dissipating much heat from energy loss. However, the junction between the current collector and the distal end of the feedthrough pin may heat up due to the high resistive nature of the unconnected surfaces. The heat generated may be high enough to heat up a surface of the feedthrough pin and a surface of the current collector in contact at the junction. The heat may cause the surfaces in contact at the junction to melt and form together in an electrically conductive and structurally solid weld.

In the example of plastic covers used in hermetically sealed IMD battery housings, a gap may be created between the feedthrough pin and the battery cover of the battery housing. The gap may be a natural result of design, or a consequence of resistance welding the feedthrough pin to the current collector. To maintain the hermiticity of the battery housing, the gap may be filled with a variety of fillers. Some fillers may include medical grade epoxy, curing resin, or other medical grade hardening jells.

Throughout the disclosure, a reference to a “feedthrough pin” refers to an elongated metallic rod formed from an electrical conductor that is configured to transmit electrical energy between a distal end and a proximal end, through a barrier in which the pin is configured to be partially disposed. The use of the term “housing” may be used to refer to a hermetically sealed housing of an IMD or any component within the IMD, which includes a hermetically sealed housing. A housing may be presumed to be hermetically sealed for purposes of this disclosure. In this disclosure, an IMD, or a variety of the IMD's components, may include a variety of feedthrough pin assemblies described or manufactured in accordance with one or more techniques of this disclosure.

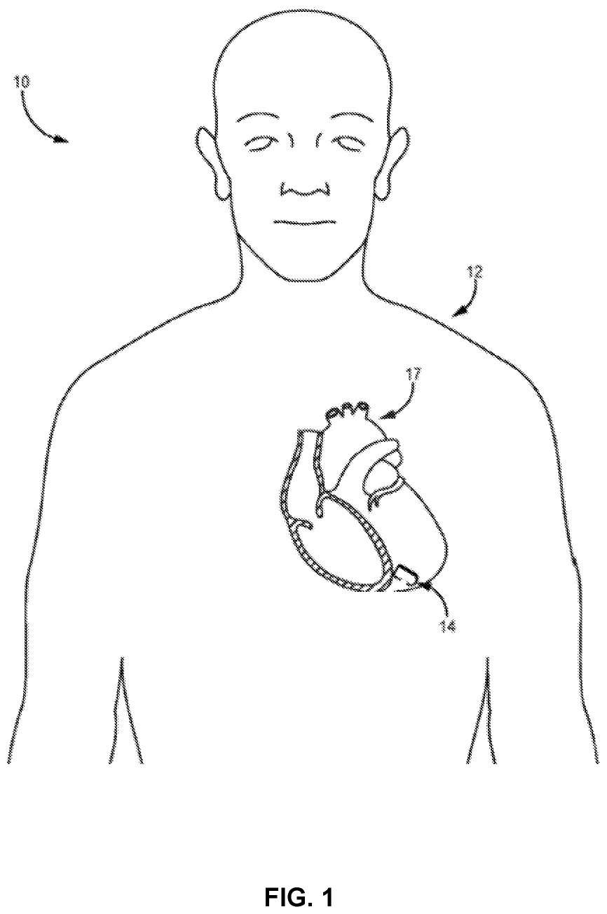

FIG. 1 is a conceptual drawing illustrating an example of a hermetically sealed Implantable Medical Device (IMD) implanted subcutaneously within a patient. The systems, devices, and techniques described in this disclosure may include example configurations of one or more feedthrough pin assemblies (not shown in FIG. 1) located within an IMD 14 (or other IMD not depicted in FIG. 1), for feeding, distributing, or transporting electrical energy between components, across a hermetically sealed barrier when implanted within a patient, as illustrated and described with respect to FIG. 1. For purposes of this description, knowledge of cardiovascular anatomy and functionality is presumed, and details are omitted except to the extent to explain the context of the techniques of this disclosure. System 10 includes IMD 14, implanted within a patient 12. IMDs may benefit from hermetically sealed housing due to the nature of exposing internal organs of a patient to foreign bodies (e.g., IMD). In some examples, sealing an IMD with a hermetically sealed housing may facilitate sterilization of the IMD. A feedthrough pin assembly may be used to feed electrical energy or electrical signals across a hermetically sealed barrier created by partially disposing the feedthrough pin into the hermetically sealed housing. The systems, devices, and techniques described herein may provide efficient feedthrough pin assemblies configured to maintain the hermeticity of the housing while allowing for the transfer of electrical energy between electrical components inside and outside of the housing. Though described in terms of a medical device system including IMD 14, in other examples, the feedthrough pin assembly techniques of this disclosure may apply to other types of devices and other industries. Examples of other types of devices may include aerospace electronics, semiconductor device packaging, actuator devices, or any other device in which hermetically sealed housing may be useful.

In some examples, IMD 14 may be implanted outside of a thoracic cavity of patient 12 (e.g., subcutaneously in the pectoral location illustrated in FIG. 1). In other examples, IMD 14 may be positioned near the sternum near or just below the level of the heart of patient 12, e.g., at least partially within the cardiac silhouette. In other examples, IMD 14 may be located in other locations on patient 12, including for monitoring and stimulation of the tibial nerve, sacral nerve, spinal cord, vagal nerve, deep brain stimulation, located at or near one or more organs or other locations. In various examples, electrical connections to tissue outside a main housing of IMD 14, may be accomplished by using one or more techniques of this disclosure.

IMD 14 may include a plurality of electrodes (not shown) and may be configured to sense a cardiac electrogram (EGM) and other bioelectrical signals via the plurality of electrodes. In some examples, electrodes may be integrated with electrical connection that utilize a feedthrough pin assembly that maintains the hermeticity of a IMD housing. In various examples, IMD 14 may represent a cardiac monitor, a defibrillator, a cardiac resynchronization pacer/defibrillator, a pacemaker, an implantable pressure sensor, a neurostimulator, glucose monitor, drug pump, pulse wave velocity measurement device or any other implantable or external medical device.

In various examples, IMD 14 may include one or more additional sensor circuits configured to sense a physiological or neurological parameter associated with patient 12. In some examples, IMD 14 may include a sensor operable to sense a body temperature of patient 12 in a location of IMD 14, or at the location of the patient where a temperature sensor coupled by a lead to IMD 14 is located (not shown in FIG. 1). In another example, IMD 14 may include a sensor configured to sense motion or position, e.g., and accelerometer, to sense steps taken by patient 12 and/or a position or a change of posture of patient 12. In various examples, IMD 14 may include a sensor that is configured to detect breaths taken by patient 12. In various examples, IMD 14 may include a sensor configured to detect heartbeats of patient 12. In various examples, IMD 14 may include a sensor that is configured to measure systemic blood pressure of patient 12 or other biological measurements. In some examples, the one or more sensors may be externally connected to electronics within a main housing of IMD14. The electrical connection to electronics within a main housing may utilize one or more feedthrough pin assembly techniques of this disclosure.

In some examples, system 10 may include one or more other sensors (not shown in FIG. 1) implanted within patient 12, that is, implanted below at least the skin level of the patient. In some examples, system 10 may include more or fewer components than depicted in FIG. 1. In some examples, system 10 may include implantable deep brain stimulators, implantable pacemaker devices, implantable neural modulators, or other IMDs, implanted within patient 12. In these examples, IMD 14 may function as a hub device for the other IMDs.

For the remainder of the disclosure, a reference to a medical device system may refer collectively to include any examples of medical device system 10, a reference to IMD 14 may refer collectively to include any examples of IMD 14, a reference to sensor circuits may refer collectively to include any examples of sensor circuits of IMD 14.

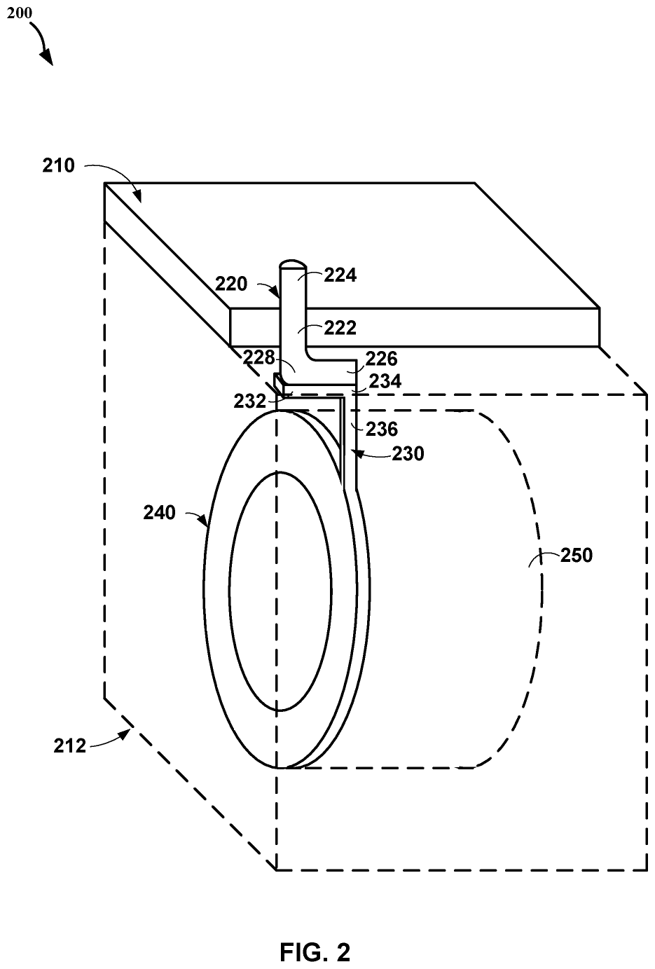

FIG. 2 is a conceptual diagram illustrating an example of a feedthrough pin assembly coupled to a battery 250, in accordance with one or more techniques of this disclosure. In some examples, a feedthrough pin assembly may include a feedthrough pin 220 having an elongated portion 222, a proximal portion 224, and a distal portion 226. Distal portion 226 may be inserted through an opening of a battery cover 210. Distal portion 226 may protrude from a distal surface of battery cover 210 while elongated portion 222 remains disposed within the opening of battery cover 210. Distal portion 226 may be configured to electrically couple to a tab 230 of a current collector 240. Current collector 240 may be configured to electrically contact battery 250 when assembled within a battery container 212. A battery housing may be defined as the battery components that include both battery container 212 and battery cover 210.

In some examples, feedthrough pin 220 may be an elongated piece of conductive metal. Distal portion 226 may be configured to have a sharpened distal end.

In some examples, battery cover 210 may include a polymer. The distal end may be configured to produce an opening in battery cover 210 by puncturing a polymer membrane of battery cover 210. In some examples, the opening in battery cover 210 may be wider than distal portion 226 of the feedthrough pin 220 being inserted through the opening. In some examples, the protruding distal portion may form a feedthrough bend 228, distal to battery cover 210. A portion of feedthrough pin 220, distal feedthrough bend 228, may align to contact a distal surface of battery cover 210. Contact with the distal surface by the distal portion of feedthrough pin 220 may prevent proximal portion 224 from being advanced into the opening in battery cover 210.

Proximal end of feedthrough pin 220 may be configured to secure feedthrough pin 220 to battery cover 210. In some examples, proximal portion 224 may be sealed to a proximal surface of battery cover 210. In some examples, proximal portion 224 may be configured for removal prior (e.g., by trimming, snipping, severing, or the like) to sealing a new proximal end to the proximal surface of battery cover 210.

In some examples, tab 230 of current collector 240 may be configured to electrically couple to distal portion 226. In some examples, both tab 230 and current collector 240 may be formed contiguously from one piece of metal. In some examples, tab 230 may include a first surface adjacent to a second surface of distal portion 226. In some examples a width of the first surface may be equal to a width of the second surface. Tab 230 may include a proximal tab portion 232 proximal a tab bend 234 and a distal tab portion 236 distal tab bend 234. In some examples, tab 230 may align with a proximal end of proximal tab portion 232 may contact feedthrough bend 228 and tab bend 234 contacts distal end of distal portion 226.

In some examples, tab 230 may be configured to electrically couple to distal portion 226 via a resistance weld. A resistance weld may be a weld formed from resistance welding a surface of distal portion 226 to a surface of proximal tab portion 232. The resistance weld may be formed from electrically contacting a first resistance welding electrode to proximal portion 224 of feedthrough pin 220. Resistance weld may be further formed by electrically contacting a second resistance welding electrode to current collector 240. The weld may be formed by applying electrical current between the first resistance welding electrode and the second resistance welding electrode. The electrical current may flow through feedthrough pin 220, current collector 240, and tab 230 heating contacting metal surfaces of distal portion 226 and proximal tab portion 232. The heating from current flow may metallically bond feedthrough pin 220 to the electrical tab 230. In some examples, current collector 240 may be configured to contact a battery terminal of battery 250. Current collector 240 may be held in contact with battery 250 by a battery container 212. In some examples, current collector 240 may include a convex surface (not illustrated in FIG. 2). Battery container 212 may be distal portion of a battery housing including battery cover 210. Battery cover 210 may be configured to hermetically seal to a proximal surface of battery container 212. Hermetically sealing battery cover 210 to battery container 212 may create a hermetically sealed housing around battery 250. In some examples, a gap between elongated portion 222 and battery cover 210 may be sealed using hermetically sealing techniques. In some examples, the gap may be exacerbated by resistance welding techniques prior to sealing battery container 212 to battery cover 210.

In some examples, battery container 212 may be attached to battery cover 210 by electrically coupling the convex surface of current collector 240 to a battery terminal. In some examples, attaching battery cover 210 to battery container 240 may further include fusing a gap between the battery cover and the battery container with a hermitic seal.

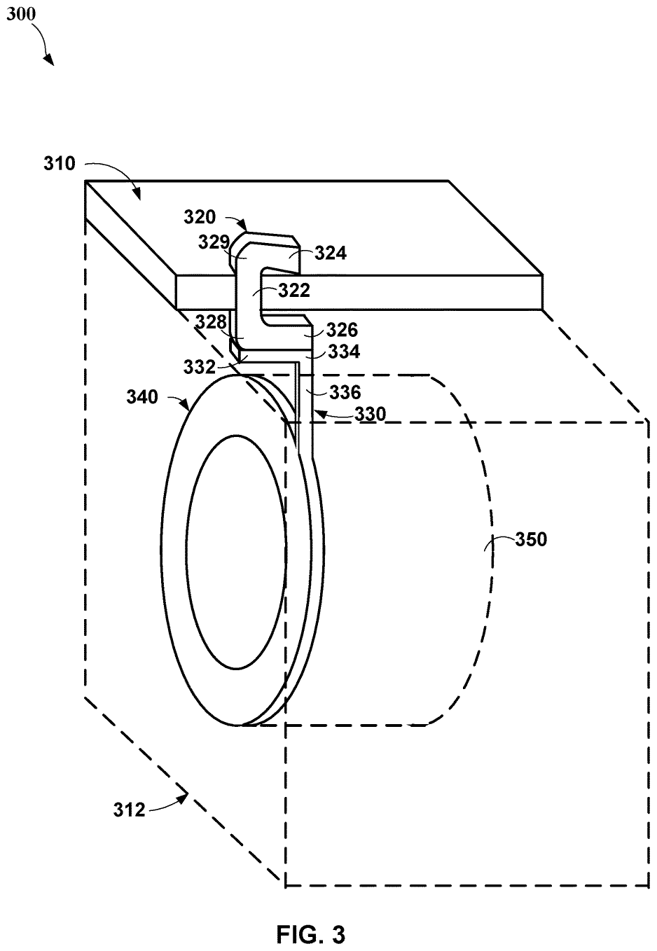

FIG. 3 is a conceptual diagram illustrating an example of a feedthrough pin assembly coupled to battery 250 and secured to a battery cover, in accordance with one or more techniques of this disclosure. In some examples, a feedthrough pin assembly may include a feedthrough pin 320. In some examples, feedthrough pin 320 and feedthrough pin 220 of FIG. 2 may be similar in shape prior to incorporation into their respective feedthrough pin assemblies. Feedthrough pin 320 may have elongated portion 322. Feedthrough pin 320 may have a distal portion 326. Feedthrough pin 320 may have a proximal portion 324. Battery cover 310 may be an example of battery cover 210 of FIG. 2. A tab 330 may be similar to tab 230 of FIG. 2.

In some examples, proximal portion 324 may be configured to bend. A securing bend 329 may be positioned proximal to battery cover 310, securing feedthrough pin 320 to battery cover 310. A portion proximal to securing bend 329, may be aligned to contact a proximal surface of battery cover 310. In some examples, securing proximal portion 324 of the feedthrough pin to the proximal surface of battery cover 310 further comprises bending proximal portion 324 of feedthrough pin 320 to contact the proximal surface of battery cover 310.

Securing bend 329 may secure feedthrough pin 320 to battery cover 310, thereby preventing distal portion 326 of feedthrough pin 320 from further advancing into the opening of battery cover 310. In some examples, proximal portion 324 may be configured to electrically connect to an electrical pad of a printed circuit board (PCB) via a solder connection or a resistance weld.

In some examples, distal portion 326 may include bend 328, aligning a distal end of distal portion 326 to point in a direction the same or relatively the same as a direction to which a proximal end of proximal portion 324 points. In some examples, both bend 328 and bend 329 may secure feedthrough pin 320 to battery cover 310. In some examples, distal portion 326 may point in a direction 180 degrees opposite, or approximately 180 degrees opposite, the direction to which the proximal end of proximal portion 324 points.

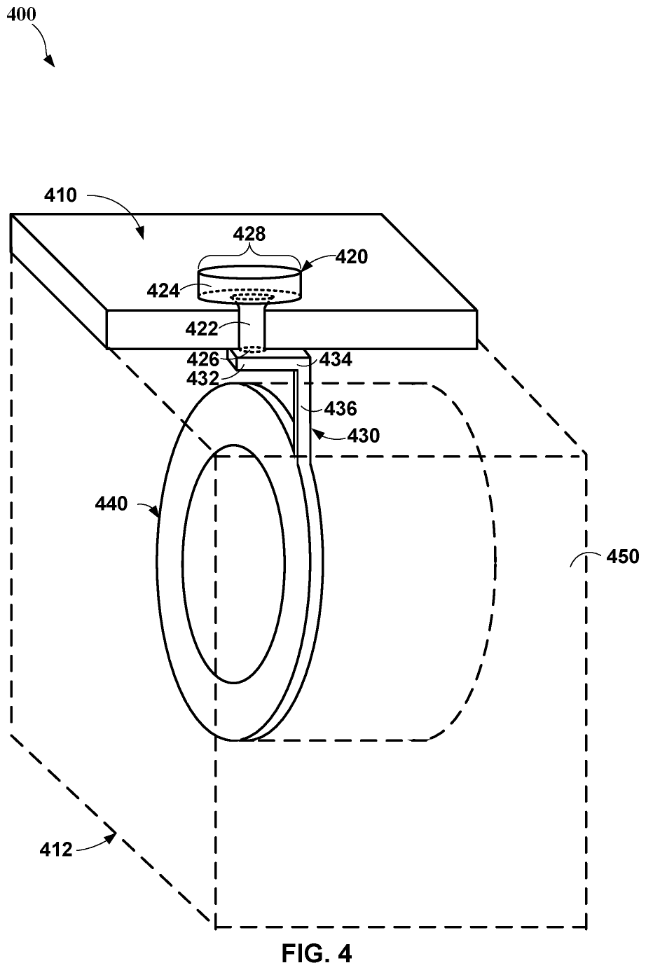

FIG. 4 is a conceptual diagram illustrating an example of a feedthrough pin assembly coupled to a battery 450 and secured to a battery with an enlarged head feedthrough pin, in accordance with one or more techniques of this disclosure. In some examples, feedthrough pin 420, feedthrough pin 320 of FIG. 3, and feedthrough pin 220 of FIG. 2 may be similar in shape prior to incorporation into their respective feedthrough pin assemblies. Feedthrough pin 420 may have elongated portion 422 an example of elongated portion 222 of FIG. 2. Feedthrough pin 420 may have a distal portion 426, an example of distal portion 226 of FIG. 2. Feedthrough pin 420 may have a proximal portion 424 an example of proximal portion 224. Battery cover 410 may be an example of battery cover 210 of FIG. 2. A tab 430 may be an example of tab 230 of FIG. 2.

In some examples, proximal portion 424 may include an enlarged portion 428 of feedthrough pin 420. Enlarged portion 428 may be larger than an opening of battery cover 410. In some examples, distal portion 426 may be inserted into the opening of battery cover 410 and advanced through the opening. In some examples, securing the proximal portion of the feedthrough pin to the proximal surface of battery cover 410 includes continuing to insert the feedthrough pin into the opening of battery cover 410 until proximal portion 424, comprising enlarged portion 428 wider than the opening, contacts the proximal surface of battery cover 410.

In some examples, battery cover 410 may be electrically insulative. In some examples, enlarged portion 428 may be one of cylindrical, pyramidal, spherical, or cubical.

In some examples, enlarged portion 428 may secure feedthrough pin 420 to battery cover 410. In some examples, proximal portion 424, having enlarged portion 428, may inhibit advancement of elongated portion 422 through the opening of battery cover 410. In some examples, enlarged portion 428 may be formed on proximal portion 424 of feedthrough pin 420 before being inserted into the opening of battery cover 410. In some examples, distal portion 426 of feedthrough pin 420 may be advanced through the opening in battery cover 410 until enlarged portion 428 abuts a proximal surface of battery cover 410. Distal portion 426 may be electrically connected to tab 430 of current collector 440. In some examples, electrically connecting tab 430 to distal portion 426 may include resistance welding feedthrough pin 420 to tab 430.

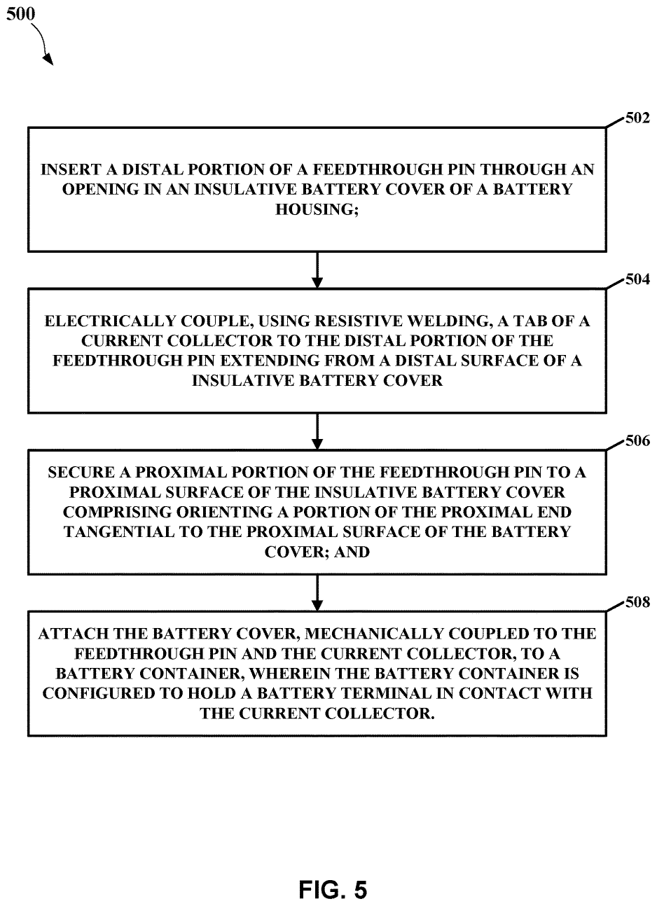

FIG. 5 is flow diagram illustrating a technique 500 of utilizing a feedthrough pin assembly in accordance with one or more techniques of the disclosure. A manufacturer may insert a distal portion of a feedthrough pin through an opening in a battery cover of a battery housing (502). In some examples, the distal portion may be any one of distal portion 226, 326, or 426 and feedthrough pin may be any one of feedthrough pin 220,320, or 420. In various examples, a battery cover (e.g., battery cover 210, 310, and 410) may be a battery. In various examples, the battery housing may include both a battery container (e.g., battery container 212, 312, and 412) and a battery cover (e.g., battery cover 210,310, and 410). In some examples, the opening in the battery cover is formed by piercing a polymer layer of the battery cover with a pointed distal end of the feedthrough pin.

In accordance with one or more techniques, a manufacturer may electrically couple, using resistance welding, a tab of a current collector to the distal portion of the feedthrough pin extending from a distal surface of a battery cover (504). In some examples, resistance welding may occur between a surface of the tab of current collector (e.g., tab 230, 330, and 430) and a surface of the distal portion (e.g., distal portion 226, 326, and 426) of the feedthrough pin. The welding may occur between the two surfaces of the tab and the distal portion that physically contact each other. In some examples, welding may cause the opening in battery cover to widen. In some examples, a gap may form between battery cover 410 and battery container 412, forming an opening in the battery housing. In some examples, applying an epoxy circumferentially around the feedthrough pin on the proximal end of the battery cover to create a hermetic seal between the feedthrough pin and the surrounding battery cover.

In some examples, resistance welding may include electrically contacting a first resistance welding electrode to the proximal end (e.g., 224,324, and 424) of the feedthrough pin (e.g., 220, 320, and 420). In some examples, resistance welding may include electrically contacting a second resistance welding electrode to the distal end (e.g., 226, 326, and 426) of the feedthrough pin. In some examples, resistance welding may include applying electrical current between the first resistance welding electrode and the second resistance welding electrode. In some examples, resistance welding may include metallically bonding the feedthrough pin to the electrical tab.

In accordance with one or more techniques, a manufacturer may secure a proximal portion of the feedthrough pin to a proximal surface of the battery cover comprising orienting a portion of the proximal end to contact the proximal surface of the battery cover (504). In some examples, the proximal portion (e.g., 224 or 324) may bend upon orienting the portion of the proximal end to contact the proximal surface of battery cover (e.g., 210, 310, or 410).

In accordance with one or more techniques, a manufacturer may attach the battery cover, mechanically coupled to the feedthrough pin and the current collector, to a battery container, wherein the battery container is configured to hold a battery terminal in contact with the current collector (508). In some examples, the battery cover (e.g., battery cover 210, 310, and/or 410) may connect to a proximal end of the battery container (e.g., battery container 212, 312, and/or 412). In various examples, the battery container may be configured to hold a current collector (e.g., current collector 240, 340, or 440) in contact with the battery (e.g., battery 250, 350, and 450) thereby creating electrical continuity from the battery to a proximal end of the feedthrough pin via the current collector.

This disclosure includes the following non-limiting examples.

Example 1. A method of utilizing a feedthrough pin assembly comprising: inserting a distal portion of a feedthrough pin through an opening in a battery cover; electrically coupling, using resistance welding, a tab of a current collector to the distal portion of the feedthrough pin extending from a distal surface of the battery cover; securing a proximal portion of the feedthrough pin to a proximal surface of the battery cover comprising orienting a portion of the proximal end to contact the proximal surface of the battery cover; and attaching the battery cover, the battery cover being mechanically coupled to the feedthrough pin, to a battery container, wherein the battery container is configured to hold a battery terminal in contact with the current collector coupled to the feedthrough pin.

Example 2. The method of example 1, wherein securing the proximal portion of the feedthrough pin to the proximal surface of the battery cover further comprises bending a proximal portion of the feedthrough pin to contact the proximal surface of the battery cover.

Example 3. The method of examples 1-2, wherein securing the proximal portion of the feedthrough pin to the proximal surface of the battery cover further comprises continuing to insert the feedthrough pin into the opening in the battery cover until a proximal portion, comprising an enlarged portion wider than the opening, contacts the proximal surface of the battery cover.

Example 4. The method of examples 1-3, wherein the opening in the battery cover is formed by piercing a polymer layer of the battery cover with a pointed distal end of the feedthrough pin.

Example 5. The method of examples 1-4, wherein the resistance welding comprises: electrically contacting a first resistance welding electrode to a proximal end of the feedthrough pin; electrically contacting a second resistance welding electrode to the tab of the current collector; applying electrical current between the first resistance welding electrode and the second resistance welding electrode; and metallically bonding the feedthrough pin to the tab of the current collector.

Example 6. The method of examples 1-5, wherein the opening in the battery cover is wider than the distal portion of the feedthrough pin being inserted through the opening.

Example 7. The method of examples 1-6, wherein fusing the gap comprises: applying an epoxy circumferentially around the feedthrough pin on the proximal end of the battery cover to create a hermetic seal between the feedthrough pin and the surrounding battery cover.

Example 8. The method of examples 1-7, wherein the battery cover comprises a polymer.

Example 9. The method of examples 1-8, wherein the tab of the current collector and the current collector are formed contiguously from one piece of metal.

Example 10. A feedthrough pin assembly for a medical device comprising: a feedthrough pin comprising a distal portion configured for insertion through an opening in a battery cover of a battery housing; a current collector comprising a tab configured to electrically couple, using resistance welding, to the distal portion of the feedthrough pin extending from a distal surface of the battery cover; and a proximal portion of the feedthrough pin configured to be secured to a proximal surface of the battery cover comprising orienting a portion of the proximal end to contact the proximal surface of the battery cover; the battery cover configured to attach to a battery container, enclosing a battery configured to contact the current collector, wherein the battery container is coupled to the feedthrough pin and current collector; wherein the battery cover, being mechanically coupled to the feedthrough pin, wherein the battery container is configured to attach to the battery cover, wherein the battery container is configured to hold a battery terminal in contact with the current collector coupled to the feedthrough pin.

Example 11. The feedthrough pin assembly of claim 10, wherein the feedthrough pin comprises a proximal portion of the feedthrough pin bent to contact the proximal surface of the battery cover, securing the proximal portion of the feedthrough pin to the proximal surface of the battery cover.

Example 12. The feedthrough pin assembly of examples 10-11, wherein the feedthrough pin comprises the proximal portion of the feedthrough pin, comprising an enlarged portion wider than the opening, configured to contact the proximal surface of the battery cover, upon continuing to insert the feedthrough pin into the opening in the battery cover.

Example 13. The feedthrough pin assembly of examples 10-12, wherein a pointed distal end of the feedthrough pin forms an opening in the battery cover by piercing a polymer layer.

Example 14. The feedthrough pin assembly of examples 11-13, wherein the resistance welding comprises: electrically contacting a first resistance welding electrode to a proximal end of the feedthrough pin; electrically contacting a second resistance welding electrode to the tab of the current collector; applying electrical current between the first resistance welding electrode and the second resistance welding electrode; and metallically bonding the feedthrough pin to the tab of the current collector.

Example 15. The feedthrough pin assembly of examples 10-14, wherein the opening in the battery cover is wider than a portion than the distal portion of the feedthrough pin being inserted through the opening.

Example 16. The feedthrough pin assembly of examples 10-15, wherein a gap may be fused comprises: applying an epoxy circumferentially around the feedthrough pin on the proximal end of the battery cover to create a hermetic seal between the feedthrough pin and the surrounding battery cover.

Example 17. The feedthrough pin assembly of examples 11-16, wherein the battery cover comprises a polymer.

Example 18. The feedthrough pin assembly of examples 11-17, wherein the tab and the current collector are formed contiguously from one piece of metal.

1. A feedthrough pin assembly for a medical device comprising:
a feedthrough pin comprising a proximal portion and a distal portion, the distal portion configured for insertion through an opening in a battery cover of a battery housing; and
a current collector comprising a tab configured to be resistance welded to and electrically couple to the distal portion of the feedthrough pin extending from a distal surface of the battery cover,
wherein the proximal portion of the feedthrough pin is configured to be secured to a proximal surface of the battery cover comprising orienting a portion of a proximal end of the feedthrough pin to contact a proximal surface of the battery cover.

2. The feedthrough pin assembly of claim 1, wherein the feedthrough pin comprises a proximal portion of the feedthrough pin bent to contact the proximal surface of the battery cover, securing the proximal portion of the feedthrough pin to the proximal surface of the battery cover.

3. The feedthrough pin assembly of claim 1, wherein the feedthrough pin comprises the proximal portion of the feedthrough pin, comprising an enlarged portion wider than the opening, configured to contact the proximal surface of the battery cover, upon continuing to insert the feedthrough pin into the opening in the battery cover.

4. The feedthrough pin assembly of claim 1, wherein a pointed distal end of the feedthrough pin forms an opening in the battery cover by piercing a polymer layer.

5. The feedthrough pin assembly of claim 1, further comprising a resistance weld between the tab and the distal portion of the feedthrough pin, the resistance weld
metallically bonding the feedthrough pin to the tab of the current collector.

6. The feedthrough pin assembly of claim 1, wherein the opening in the battery cover is wider than a portion than the distal portion of the feedthrough pin being inserted through the opening.

7. The feedthrough pin assembly of claim 1, further comprising a fused gap,
the fused gap comprising an epoxy circumferentially applied around the feedthrough pin on a proximal end of the battery cover to create a hermetic seal between the feedthrough pin and the battery cover.

8. The feedthrough pin assembly of claim 1, wherein the battery cover comprises a polymer.

9. The feedthrough pin assembly of claim 1, wherein the tab and the current collector are formed contiguously from one piece of metal.

10. The feedthrough pin assembly of claim 1, wherein the medical device comprises an implantable medical device.

11. The feedthrough pin assembly of claim 1, wherein the battery cover is configured to attach to a battery container, enclosing a battery configured to contact the current collector, wherein the battery container is configured to be coupled to the feedthrough pin and current collector, wherein the battery cover is configured to be mechanically coupled to the feedthrough pin, wherein the battery container is configured to attach to the battery cover, and wherein the battery container is configured to hold a battery terminal in contact with the current collector coupled to the feedthrough pin.

12. A method of utilizing a feedthrough pin assembly comprising:
inserting a distal portion of a feedthrough pin through an opening in a battery cover;
electrically coupling, using resistance welding, a tab of a current collector to the distal portion of the feedthrough pin extending from a distal surface of the battery cover;
securing a proximal portion of the feedthrough pin to a proximal surface of the battery cover comprising orienting a portion of the proximal portion of the feedthrough pin to contact the proximal surface of the battery cover; and
attaching the battery cover, the battery cover being mechanically coupled to the feedthrough pin, to a battery container, wherein the battery container is configured to hold a battery terminal in contact with the current collector coupled to the feedthrough pin.

13. The method of claim 12, wherein securing the proximal portion of the feedthrough pin to the proximal surface of the battery cover further comprises bending a proximal portion of the feedthrough pin to contact the proximal surface of the battery cover.

14. The method of claim 12, wherein securing the proximal portion of the feedthrough pin to the proximal surface of the battery cover further comprises continuing to insert the feedthrough pin into the opening in the battery cover until a proximal portion, comprising an enlarged portion wider than the opening, contacts the proximal surface of the battery cover.

15. The method of claim 12, wherein the opening in the battery cover is formed by piercing a polymer layer of the battery cover with a pointed distal end of the feedthrough pin.

16. The method of claim 12, wherein the resistance welding comprises:
electrically contacting a first resistance welding electrode to a proximal end of the feedthrough pin;
electrically contacting a second resistance welding electrode to the tab of the current collector;
applying electrical current between the first resistance welding electrode and the second resistance welding electrode; and
metallically bonding the feedthrough pin to the tab of the current collector.

17. The method of claim 12, wherein the opening in the battery cover is wider than the distal portion of the feedthrough pin being inserted through the opening.

18. The method of claim 12, further comprising applying an epoxy circumferentially around the feedthrough pin on a proximal end of the battery cover to create a hermetic seal between the feedthrough pin and the battery cover.

19. The method of claim 12, wherein the battery cover comprises a polymer.

20. A medical device comprising:
a battery housing comprising a battery cover: and
a feedthrough pin assembly, the feedthrough pin assembly comprising:
a feedthrough pin comprising a proximal portion and a distal portion, the distal portion inserted through an opening in the battery cover of the battery housing; and
a current collector comprising a tab that is resistance welded to and electrically couples to the distal portion of the feedthrough pin extending from a distal surface of the battery cover,
wherein the proximal portion of the feedthrough pin is secured to a proximal surface of the battery cover wherein a portion of a proximal end of the feedthrough pin is in contact with a proximal surface of the battery cover, wherein the battery cover is attached to a battery container, enclosing a battery configured to contact the current collector, wherein the battery container is coupled to the feedthrough pin and current collector, wherein the battery cover is mechanically coupled to the feedthrough pin, wherein the battery container is attached to the battery cover, and wherein the battery container holds a battery terminal in contact with the current collector coupled to the feedthrough pin.
